(19) United States
(12) Patent Application Publication
(10) Pub. No.: US 2015/0094575 A1
(43) Pub. Date: Apr. 2, 2015

(54) METHOD AND DEVICE FOR TRACKING CONTRAST MEDIUM

(71) Applicant: Shenyang Neusoft Medical Systems Co., Ltd., Shenyang (CN)

(72) Inventors: Shanshan LOU, Shenyang (CN); Ling PANG, Shenyang (CN)

(73) Assignee: Shenyang Neusoft Medical Systems Co., Ltd., Shenyang (CN)

(21) Appl. No.: 14/142,948

(22) Filed: Dec. 30, 2013

(30) Foreign Application Priority Data
Sep. 27, 2013 (CN) 201310449979.6

(51) Int. Cl. A61M 5/00; A61B 6/00

(52) U.S. Cl. CPC A61M 5/007; A61B 6/481
USPC 600/431

(57) ABSTRACT

A method and a device for tracking a contrast medium by scanning are provided. The method includes: obtaining a relationship between a contrast medium concentration and time according to concentration values of the contrast medium obtained by previous scannings; predicting a time interval from a current time point to a time point when a target concentration value reaches according to the relationship between the contrast medium concentration and time; and starting a routine examination at the end of the time interval. In the present disclosure, a routine examination may be performed in time.

### CROSS-REFERENCE TO RELATED APPLICATIONS

The present application claims priority to Chinese patent application No. 201310449979.6, filed on Sep. 27, 2013, and entitled “METHOD AND DEVICE FOR TRACKING CONTRAST MEDIUM”, and the entire disclosure of which is incorporated herein by reference.

### FIELD OF THE DISCLOSURE

The present disclosure generally relates to medical diagnosis device field, and more particularly, to a method and a device for tracking a contrast medium by scanning.

### BACKGROUND OF THE DISCLOSURE

At present, in a CT (Computed Tomography) system where a contrast medium is generally used to acquire images, the contrast medium may be tracked periodically before image acquisition, to detect whether the concentration value of the contrast medium reaches a target value required for a routine examination. When the concentration value reaches the target value, the tracking of the contrast medium will be terminated and the routine examination may be started immediately.

For example, a contrast medium scanning in a scanning period may include: scanning an object; obtaining a location image of the object; selecting a ROI (Region Of Interest) from the location image; measuring a CT value of the contrast medium in the ROI; and determining whether the CT value reaches a target CT value; if yes, the concentration value of the contrast medium is determined to have reached the target concentration value required for the routine examination, so the tracking of the contrast medium is stopped for the routine examination immediately; if no, the concentration value of the contrast medium is determined to have not yet reached the target concentration value required for the routine examination, so a next round of scanning will be performed on the contrast medium (i.e., repeating the above processes).

For a timely detection of the state of the target concentration of the contrast medium exactly after the concentration value of the contrast medium reaches the target concentration value and thus for a timely routine examination, the scanning period is set relatively short.

However, it is found out that, in the conventional technologies, although the scanning period is reduced, it is inevitably that the concentration value of a contrast medium reaches the target concentration value long before a next scanning period (i.e., before the next scanning is performed). So the target concentration value of the contrast medium can only be detected in the next scanning, which cannot ensure a timely routine examination.

Besides, the reduction of the scanning period may inevitably result in an increased number of scannings, which may cause a waste of scanning dosage and increase radiation dosage delivered to a patient.

### SUMMARY

Embodiments of the present disclosure provide a method and device for tracking contrast medium by scanning to ensure a timely routine examination. Further, a scanning dosage may be saved and radiation dosage delivered to a patient may be avoided.

In an embodiment, a method for tracking a contrast medium by scanning may be provided, including:

obtaining a relationship between a contrast medium concentration and time, according to concentration values of the contrast medium obtained by previous scannings;

predicting a time interval from a current time point to a time point when a target concentration value reaches according to the relationship between the contrast medium concentration and time; and

starting a routine examination at the end of the time interval.

Optionally, the method may further include: after predicting the time interval from the current time point to the time point when the target concentration value reaches according to the relationship between the contrast medium concentration and time, determining whether the time interval is shorter than a predetermined time threshold; if yes, stopping scanning the contrast medium; or else, continuing performing a next scanning.

Optionally, the predetermined time threshold may include a scanning period or a plurality of scanning periods.

Optionally, the target concentration value may be a concentration value required for the routine examination or any other predetermined concentration value.

Optionally, obtaining a relationship between a contrast medium concentration and time according to concentration values of the contrast medium obtained by previous scannings may include: obtaining concentration values of the contrast medium obtained by previous scannings; performing fitting to the concentration values of the contrast medium obtained by previous scannings; and generating a fitting function between the contrast medium concentration and time, wherein an independent variable of the fitting function is time and a dependent variable of the fitting function is the contrast medium concentration.

Optionally, the method may further include: before predicting the time interval from the current time point to the time point when the target concentration value reaches according to the relationship between the contrast medium concentration and time, calculating a difference between a current concentration value of the contrast medium and a concentration value of the contrast medium in a previous scanning; and determining whether the difference is greater than or equal to a predetermined concentration threshold. If yes, the relationship between the contrast medium concentration and time is obtained according to the concentration values of the contrast medium obtained by previous scannings. Optionally, if the difference is less than the predetermined concentration threshold, the next scanning may be performed.

In an embodiment, a device for tracking a contrast medium by scanning may be provided, including:

a prediction unit, adapted to obtain a relationship between a contrast medium concentration and time according to concentration values of the contrast medium obtained by previous scannings;

a time determination unit, adapted to predict a time interval from a current time point to a time point when a target concentration value reaches according to the relationship between the contrast medium concentration and time; and

a routine examination starting unit, adapted to start a routine examination at the end of the time interval.

Optionally, the device may further include: a first determination unit, adapted to determine whether the time interval from the current time point to the time point when the target concentration value reaches is shorter than a predetermined time threshold after the time interval is predicted according to the relationship between the contrast medium concentration and time; a scanning termination unit, adapted to stop scanning the contrast medium if the time interval is determined to be shorter than the predetermined time threshold; and a first scanning continuing unit, adapted to continue performing a next scanning if the time interval is determined to be longer than or equal to the predetermined time threshold.

Optionally, the predetermined time threshold may include a scanning period or a plurality of scanning periods.

Optionally, the target concentration value may be a concentration value required for the routine examination or any other predetermined concentration value.

Optionally, the prediction unit may include: an obtaining unit, adapted to obtain concentration values of the contrast medium in previous scannings; and a fitting unit, adapted to perform fitting to the concentration values of the contrast medium in previous scannings and generate a fitting function between the contrast medium concentration and time, wherein an independent variable of the fitting function is time and a dependent variable of the fitting function is the contrast medium concentration.

Optionally, the device may further include: a difference calculation unit, adapted to calculate a difference between a current concentration value of the contrast medium and a concentration value of the contrast medium in a previous scanning before the time interval from the current time point to the time point when the target concentration value reaches is determined according to the relationship between the contrast medium concentration and time; a second determination unit, adapted to determine whether the difference is greater than or equal to a predetermined concentration threshold. Optionally, if the difference is determined to be greater than or equal to the predetermined concentration threshold, the prediction unit may be adapted to obtain a relationship between a contrast medium concentration and time according to concentration values of the contrast medium obtained by previous scannings.

Optionally, the device may further include a second scanning continuing unit, adapted to continue performing a next scanning when the difference is determined to be less than the predetermined concentration threshold.

Compared with the conventional solutions, the present disclosure may have following advantages.

In the present disclosure, by obtaining the relationship between the contrast medium concentration and time, a time interval from a current time point to a time point when a target concentration value reaches may be predicted accurately. A routine examination may be started at the end of the time interval, which ensures a timely routine examination.

Further, a relatively long scanning period can be set in the disclosure, which thus saves a scanning dosage and avoids radiation dosage delivered to a patient.

### BRIEF DESCRIPTION OF THE DRAWINGS

In order to clarify the disclosure and advantages of the present disclosure, accompanying drawings used in description of embodiments of the present disclosure will be described simply. Obviously, drawings described below are only illustrative and those skilled in the art can obtain other drawings based on these drawings without creative works.

FIG. 1 schematically illustrates a flow chart of a method for tracking a contrast medium by scanning according to a first embodiment of the present disclosure;

FIG. 2 schematically illustrates a flow chart of a method for tracking a contrast medium by scanning according to a second embodiment of the present disclosure;

FIG. 3 schematically illustrates an example of a CT value curve of a contrast medium according to an embodiment of the present disclosure;

FIG. 4 schematically illustrates a flow chart of a method for tracking a contrast medium by scanning according to a third embodiment of the present disclosure;

FIG. 5 schematically illustrates a flow chart of a method for tracking a contrast medium by scanning according to a fourth embodiment of the present disclosure; and

FIG. 6 schematically illustrates a block diagram of a device for tracking a contrast medium by scanning according to a fifth embodiment of the present disclosure.

### DETAILED DESCRIPTION OF THE DISCLOSURE

In order to clarify the objects, characteristics and advantages of the disclosure, embodiments of present disclosure will be described in detail in conjunction with accompanying drawings.

### First Embodiment

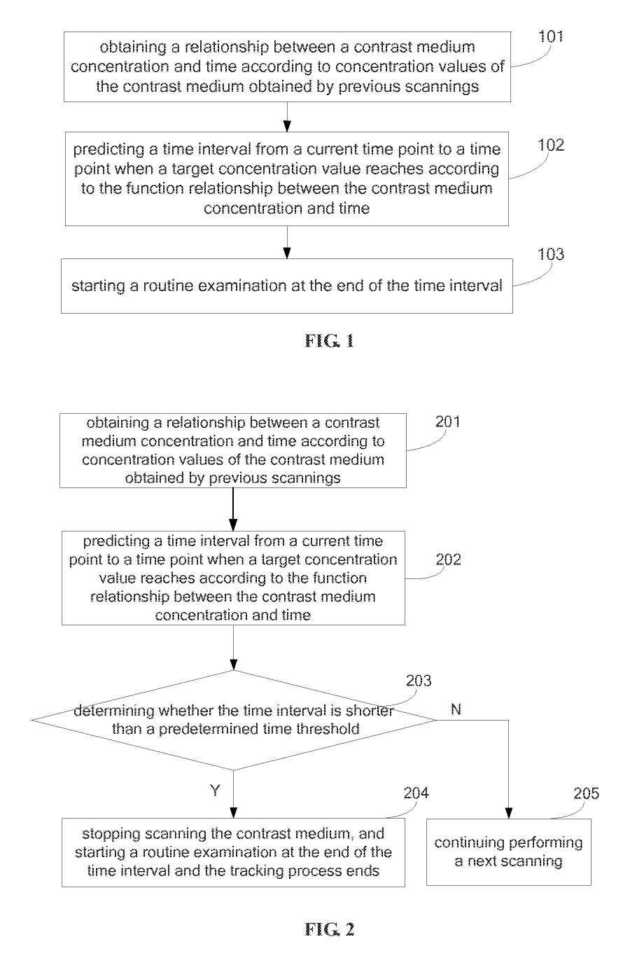

FIG. 1 schematically illustrates a flow chart of a method for tracking a contrast medium by scanning according to a first embodiment of the present disclosure. The method includes steps 101, 102 and 103.

In step 101, obtaining a relationship between a contrast medium concentration and time according to concentration values of the contrast medium obtained by previous scannings.

It should be noted that, obtaining a relationship between a contrast medium concentration and time may include: predicting contrast medium concentration values which varies with time. In some embodiments, step 101 may include: obtaining concentration values of the contrast medium obtained by previous scannings; performing fitting to the concentration values of the contrast medium obtained by previous scannings; and generating a fitting function between the contrast medium concentration and time, wherein an independent variable of the fitting function is time and a dependent variable of the fitting function is the contrast medium concentration.

For example, assuming that a current scanning is a fifth one, fitting may be performed to the five concentration values of the contrast medium from a first scanning to the fifth scanning. In some embodiments, a LSF (Least Squares Fitting) method may be employed, where a basis function may be a polynomial function, such as y=aₙxⁿ+aₙ₋₁xⁿ⁻¹+ . . . +a₁x+a₀, an exponential function, such as y=a*c^{kx-b}+d, or other fitting functions. For example, the concentration values of the contrast medium from the first scanning to the fifth scanning are respectively 20, 25, 33, 40 and 50. The product of the number of scanning times and a time period between two successive scannings is time. An independent variable of the fitting function is time and a dependent variable of the fitting function is the contrast medium concentration.

In step 102, predicting a time interval from a current time point to a time point when a target concentration value reaches according to the function relationship between the contrast medium concentration and time.

After obtaining the current concentration value and the target concentration value of the contrast medium, the time interval from the current time point to the time point when the target concentration value reaches may be predicted according to the function relationship between the contrast medium concentration and time.

In some embodiments, the target concentration value may be a target concentration value required for a routine examination or any other predetermined concentration value. In some embodiments, if a routine examination is expected to start before the target concentration value required for the routine examination reaches, the target concentration value may be set to be any value smaller than the target concentration value required for the routine examination. In some embodiments, if a user wants to delay a routine examination, the target concentration value may be set to be any value greater than the target concentration value required for the routine examination.

In step 103, starting a routine examination at the end of the time interval.

In practice, a concentration value of a contrast medium may be reflected by a CT value of the contrast medium. Therefore, the above mentioned concentration value may be replaced with CT value. That is to say, in step 101, the relationship between the contrast medium concentration and time may be replaced with a relationship between the CT value of the contrast medium and time according to CT values of the contrast medium obtained by previous scannings; and in step 102, a time interval from the current time point to a time point when a target CT value reaches may be determined according to the function relationship between the CT value of the contrast medium and time.

In conclusion, compared with the conventional technologies, the present disclosure has following advantages.

By obtaining the relationship between the contrast medium concentration and time, a time interval from a current time point to a time point when a target concentration value reaches may be predicted accurately. A routine examination may be started at the end of the time interval, which ensures a timely routine examination.

Further, a relatively longer scanning period can be set, which thus saves scanning dosage and avoids radiation dosage delivered to a patient.

### Second Embodiment

In the first embodiment, the more the number of scanning times is, the greater the original data amount (i.e., the amount of the concentration values of the contrast medium obtained by previous scannings) is, the more accurate the fitting result (i.e., the relationship between the contrast medium concentration and time) is, and thus a more timely routine examination can be ensured. However, the scanning dosage and the radiation dosage delivered to a patient usually increase with the number of scanning times. Differently from the first embodiment, in the second embodiment, by controlling the number of scanning times effectively, the accuracy of prediction results, the scanning dosage and the radiation dosage delivered to a patient may be controlled according to a user's requirements.

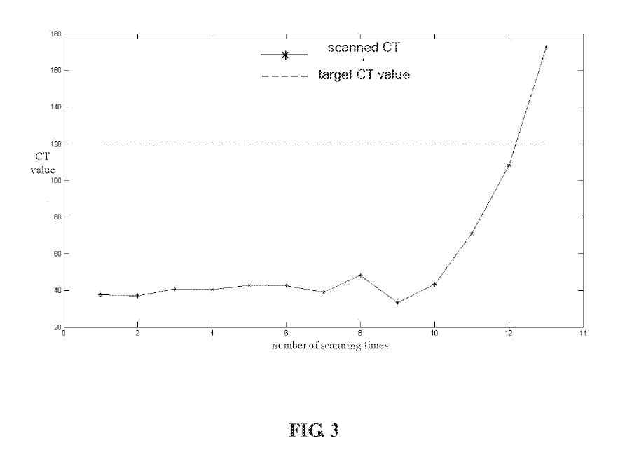

FIG. 2 schematically illustrates a flow chart of a method for tracking a contrast medium by scanning according to the second embodiment of the present disclosure. The method includes steps 201 to 205.

In step 201, obtaining a relationship between a contrast medium concentration and time according to concentration values of the contrast medium obtained by previous scannings.

In step 202, predicting a time interval from a current time point to a time point when a target concentration value reaches according to the function relationship between the contrast medium concentration and time.

The steps 201 and 202 may be performed similarly to the steps 101 and 102 in the first embodiment, which will not be described in detail.

In step 203, determining whether the time interval is shorter than a predetermined time threshold, if yes, proceeding to step 204; or else, proceeding to step 205.

In step 204, stopping scanning the contrast medium, and starting a routine examination at the end of the time interval and the tracking process ends.

In step 205, continuing performing a next scanning.

In some embodiments, the predetermined time threshold may be a scanning period or a plurality of scanning periods according to a user's requirement. For example, if a user requires a high accuracy of the prediction result and care little for scanning dosage and radiation dosage delivered to a patient, the predetermined time threshold may be set to be one scanning period. In this case, when the time interval from the current time point to the time point when the target concentration value reaches is shorter than the scanning period, the scanning may be terminated. That is to say, before the next scanning, the concentration value of the contrast medium can reach the target concentration value and the scanning ends at the current time point. Therefore, one scanning, i.e., the next scanning, is saved and the total number of scanning times are relatively high, which ensures a high accuracy of the prediction result. Otherwise, if a user cares little for a high accuracy of the prediction result, but is strict in the scanning dosage and radiation dosage delivered to a patient, the predetermined time threshold may include a plurality of scanning periods and the number of the plurality of scanning periods may depend on requirements of the user.

It should be noted that, the time interval from the current time point to the time point when the target concentration value reaches equals to a product of the number of scanning times from the current time point to the time point when the target concentration value reaches and the scanning period. Therefore, the method may be performed with following steps: step 201, obtaining a relationship between a contrast medium concentration and number of scanning times according to concentration values of the contrast medium obtained in previous scannings: step 202, determining the number of scanning times from a current time point to a time point when a target concentration value reaches according to the relationship between the contrast medium concentration and the number of scanning times; and step 203, determining whether the number of scanning times from the current time point to the time point when the target concentration value reaches is greater than or equal to a predetermined threshold of times, if yes, scanning is stopped; or else, a next scanning is continued.

From the second embodiment, compared with the conventional technologies, the present disclosure has following advantages.

By obtaining the relationship between the contrast medium concentration and time, a time interval from a current time point to a time point when a target concentration value reaches may be predicted accurately. A routine examination may be started at the end of the time interval, which ensures a timely routine examination.

Further, a relatively longer scanning period can be set, which thus saves scanning dosage and avoids radiation dosage delivered to a patient.

Compared with the first embodiment, in the second embodiment, the number of scanning times effectively, the accuracy of prediction results, the scanning dosage and radiation dosage delivered to a patient may be controlled according to a user's requirements.

### Third Embodiment

In the first and second embodiments, in each scanning, the relationship between a contrast medium concentration and time may be obtained according to concentration values of the contrast medium obtained in previous scannings, and a time interval is predicted according to the relationship between the contrast medium concentration and time. However, as shown in FIG. 3, in an initial stage, a concentration value of a contrast medium generally increases slowly, substantially within a relatively low range, and impossibly reaches a target concentration value. In a later stage, the concentration value of the contrast medium may increase rapidly and reach the target concentration value. Therefore, to save the cost caused by the obtaining and predicting step, in some embodiments, a relationship between a contrast medium concentration and time is not necessary in each scanning but is only necessary in the later stage where the concentration value increases rapidly.

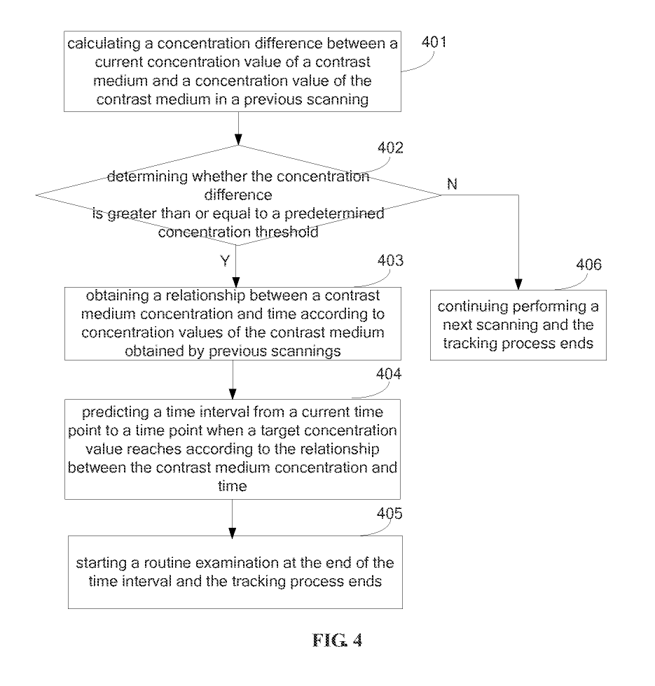

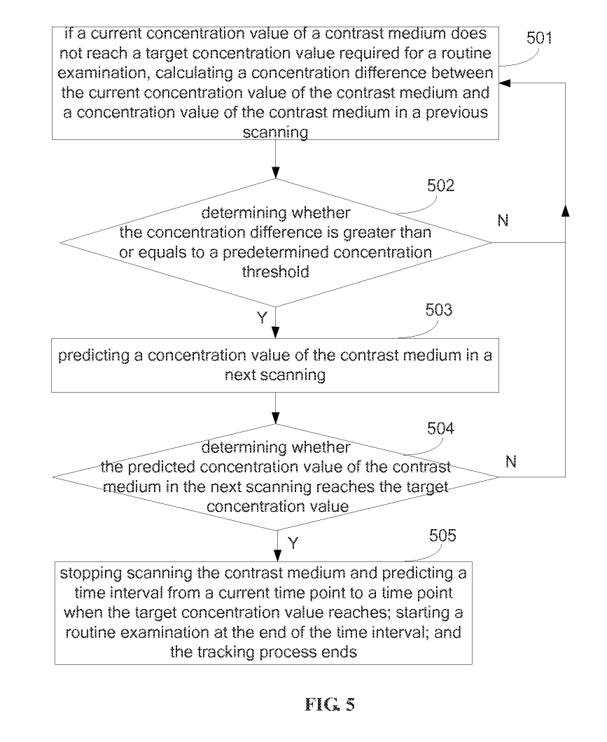

Differently from the first embodiment, in the third embodiment, before obtaining the relationship between the contrast medium concentration and time, whether performing the prediction need to be determined. If yes, the prediction may be performed; if no, the prediction may not be performed and a next scanning may be performed. FIG. 4 schematically illustrates a flow chart of a method for tracking a contrast medium by scanning according to a third embodiment of the present disclosure, the method including steps 401 to 406.

In step 401, calculating a concentration difference between a current concentration value of a contrast medium and a concentration value of the contrast medium in a previous scanning.

In step 402, determining whether the concentration difference is greater than or equal to a predetermined concentration threshold, if yes, proceeding to step 403; or else, proceeding to step 406.

In some embodiments, a CT difference between a current CT value of the contrast medium and a CT value of the contrast medium in a previous scanning may be calculated to reflect the concentration difference.

In step 403, obtaining a relationship between a contrast medium concentration and time according to concentration values of the contrast medium obtained by previous scannings.

In step 404, predicting a time interval from a current time point to a time point when a target concentration value reaches according to the relationship between the contrast medium concentration and time.

In step 405, starting a routine examination at the end of the time interval and the tracking process ends.

In step 406, continuing performing a next scanning and the tracking process ends.

The steps 403 to 405 may be performed similarly to the steps 101 to 103, which will not be described in detail.

From the third embodiment, compared with the conventional technologies, the present disclosure has following advantages.

By obtaining the relationship between the contrast medium concentration and time, a time interval from a current time point to a time point when a target concentration value reaches may be predicted accurately. A routine examination may be started at the end of the time interval, which ensures a timely routine examination.

Further, a relatively longer scanning period can be set, which thus saves scanning dosage and avoids radiation dosage delivered to a patient.

Compared with the first embodiment, in the third embodiment, cost caused by the prediction may be saved.

### Fourth Embodiment

Hereafter, a process for tracking contrast medium by scanning is described in detail. FIG. 5 schematically illustrates a flow chart of a method for tracking a contrast medium by scanning according to a fourth embodiment of the present disclosure, the method including steps 501 to 505.

In step 501, if a current concentration value of a contrast medium does not reach a target concentration value required for a routine examination, calculating a concentration difference between the current concentration value of the contrast medium and a concentration value of the contrast medium in a previous scanning.

In step 502, determining whether the concentration difference is greater than or equals to a predetermined concentration threshold, if yes, proceeding to step 503; or else, back to step 501.

In some embodiments, a CT difference between a current CT value of the contrast medium and a CT value of the contrast medium a previous scanning may be calculated to reflect the concentration difference.

In step 503, predicting a concentration value of the contrast medium in a next scanning.

In step 504, determining whether the predicted concentration value of the contrast medium in the next scanning reaches the target concentration value, if yes, proceeding to step 505; or else, back to step 503 to perform a next scanning.

In some embodiments, a CT value of the contrast medium may be calculated to reflect the concentration value of the contrast medium, and determining whether a concentration value of the contrast medium reaches the target concentration value equals to determining whether the CT value of the contrast medium reaches a target CT value. Therefore, in some embodiments, step 503 may include: predicting a CT value of the contrast medium in the next scanning; and fitting the CT values of the contrast medium from the first scanning to the current scanning, wherein a dependent variable of a fitting function is the CT value of the contrast medium. And step 504 may include: determining whether the CT value of the contrast medium in a next scanning can reach a target CT value. It should be noted that, ‘reach’ denotes to ‘be greater than’ or ‘equal to’.

In step 505, stopping scanning the contrast medium and predicting a time interval from a current time point to a time point when the target concentration value reaches; starting a routine examination at the end of the time interval; and the tracking process ends.

In some embodiments, the time interval from the current time point to the time point when the target CT value reaches may be predicted to reflect the time interval from the current time point to the time point when the target concentration value reaches.

Hereafter, FIG. 3 is described as an example. Assuming a target CT value of a contrast medium is 120 and a scanning period is 2 s. In conventional technologies, when a CT value of the contrast medium in a twelfth scanning does not reach 120, a thirteenth scanning should be performed. However, a CT value in the thirteenth scanning is much greater than the target CT value. In the present disclosure, if it is predicted that the CT value can reach the target CT value at about 0.5 s after the twelfth scanning, the thirteenth scanning will not be performed. A routine examination may be started at about 0.5 s after the twelfth scanning, thereby avoiding extra radiation caused by the thirteenth scanning and improving the accuracy of tracking the contrast medium.

From the fourth embodiment, compared with the conventional technologies, the present disclosure has following advantages.

Before performing a next scanning, a concentration value of the contrast medium in the next scanning may be predicted. If the concentration value reaches a target concentration value in the next scanning, the concentration value may have reached the target concentration value before the next scanning. To avoid extra radiation, the next scanning may not be performed immediately. Instead, a time interval from a current time point to a time point when the target concentration value reaches is predicted and a routine examination is started at the end of the time interval, which avoids one scanning and further avoids radiation dosage caused thereby.

Further, because of the prediction process, extra radiation can be avoided and a scanning period may be set to be relatively long, so that scanning dosage may be saved.

### Fifth Embodiment

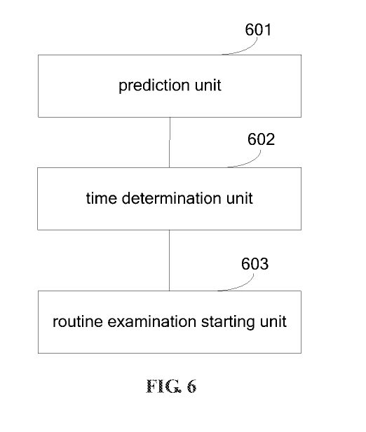

Accordingly, in one embodiment, a device for tracking a contrast medium by scanning may be provided. FIG. 6 schematically illustrates a block diagram of a device for tracking a contrast medium by scanning according to a fifth embodiment of the present disclosure. The device includes a prediction unit 601, a time determination unit 602 and a routine examination starting unit 603. Detailed structures in the device and connections of the structures may be described in conjunction with an operational principle of the device.

The prediction unit 601 is adapted to obtain a relationship between a contrast medium concentration and time according to concentration values of the contrast medium obtained by previous scannings.

The time determination unit 602 is adapted to predict a time interval from a current time point to a time point when a target concentration value reaches according to the relationship between the contrast medium concentration and time.

The routine examination starting unit 603 is adapted to start a routine examination at the end of the time interval.

In some embodiments, the target concentration value may be a concentration value required for the routine examination or any other predetermined concentration value.

In some embodiments, the device may further include: a first determination unit, adapted to determine whether the time interval from the current time point to the time point when the target concentration value reaches is shorter than a predetermined time threshold after the time interval is predicted according to the relationship between the contrast medium concentration and time; a scanning termination unit, adapted to stop scanning the contrast medium if the time interval is determined to be shorter than the predetermined time threshold; and a first scanning continuing unit, adapted to continue performing a next scanning if the time interval is determined to be longer than or equal to the predetermined time threshold.

In some embodiments, the predetermined time threshold may be a scanning period or a plurality of scanning periods.

In some embodiments, the prediction unit 601 may include: an obtaining unit, adapted to obtain concentration values of the contrast medium in previous scannings; and a fitting unit, adapted to perform fitting to the concentration values of the contrast medium in previous scannings and generate a fitting function between the contrast medium concentration and time, wherein an independent variable of the fitting function is time and a dependent variable of the fitting function is the contrast medium concentration.

In some embodiments, the device may further include: a difference calculation unit, adapted to calculate a difference between a current concentration value of the contrast medium and a concentration value of the contrast medium in a previous scanning before the time interval from the current time point to the time point when the target concentration value reaches is determined according to the relationship between the contrast medium concentration and time; a second determination unit, adapted to determine whether the difference is greater than or equal to a predetermined concentration threshold. In some embodiments, if the difference is determined to be greater than or equal to the predetermined concentration threshold, the prediction unit may be adapted to obtain a relationship between a contrast medium concentration and time according to concentration values of the contrast medium obtained by previous scannings.

In some embodiments, the device may further include: a second scanning continuing unit, adapted to continue performing a next scanning when the difference is determined to be less than the predetermined concentration threshold.

From the fifth embodiment, compared with the conventional technologies, the present disclosure has following advantages.

By obtaining the relationship between the contrast medium concentration and time, a time interval from a current time point to a time point when a target concentration value reaches may be predicted accurately. A routine examination may be started at the end of the time interval, which ensures a timely routine examination.

Further, a relatively longer scanning period can be set, which thus saves scanning dosage and avoids radiation dosage delivered to a patient.

Further, cost caused by the prediction may be saved.

Further, the number of scanning times effectively, the accuracy of prediction results, the scanning dosage and radiation dosage delivered to a patient may be controlled according to a user's requirements.

Those skilled in the art could understand that, working procedures of systems, devices and units described above may be referring to methods provided in embodiments above, which are not illustrated in detail here for brief.

It should be noted that systems, devices and methods provided in embodiments of the present disclosure are merely examples, which can be implemented in alternative ways. For example, system embodiments described above are only illustrative. Divisions of devices and units in the system are only examples for dividing logic functions. Other divisions may be employed in practice. For example, several units or components may be combined or integrated in another system, or some features can be ignored or not performed. Besides, couplings, direct couplings or communication connections between units may be realized by some interfaces. Indirect couplings or communication connections between devices or between units may be electrical, mechanical or of other forms.

Units described as separated components may be separated physically or not. Components illustrated as units may be physical units or not, that is, they may be disposed in a same place or distributed in a plurality of network cells. Some or all of the units may be selected according to practical requirements to implement embodiments of the present disclosure.

Besides, units in embodiments of the present disclosure may be integrated in one processing unit or be separated physically, or at least two units thereof are integrated in one processing unit. The integrated units may be implemented by hardware or software.

It should be noted that, those skilled in the art may understand all or some of the processes in the methods described above can be realized by using computer programs to instruct corresponding hardware. The programs may be stored in a readable storage medium in a computer. When the programs are implemented, the processes in the methods in the above embodiments may be performed. The readable storage medium may be diskette, CD (Compact Disc), ROM (Read-Only Memory), RAM (Random Access Memory) or the like.

A method and a device for tracking a contrast medium by scanning are described. Although the present disclosure has been disclosed above with reference to preferred embodiments thereof, it should be understood that the disclosure is presented by way of example only, and not limitation. Those skilled in the art can modify and vary the embodiments without departing from the spirit and scope of the present disclosure.

What is claimed is:

1. A method for tracking a contrast medium by scanning, comprising:
obtaining a relationship between a contrast medium concentration and time according to concentration values of the contrast medium obtained by previous scannings;
predicting a time interval from a current time point to a time point when a target concentration value reaches according to the relationship between the contrast medium concentration and time; and
starting a routine examination at the end of the time interval.

2. The method according to claim 1, further comprising:
after predicting the time interval from the current time point to the time point when the target concentration value reaches according to the relationship between the contrast medium concentration and time, determining whether the time interval is shorter than a predetermined time threshold; if yes, stopping scanning the contrast medium; or else, continuing performing a next scanning.

3. The method according to claim 1, wherein the target concentration value is a concentration value required for the routine examination or any other predetermined concentration value.

4. The method according to claim 2, wherein the predetermined time threshold comprises a scanning period or a plurality of scanning periods.

5. The method according to claim 1, wherein obtaining a relationship between a contrast medium concentration and time according to concentration values of the contrast medium obtained by previous scannings comprises:
obtaining concentration values of the contrast medium obtained by previous scannings; and
performing fitting to the concentration values of the contrast medium obtained by previous scannings to generate a fitting function between the contrast medium concentration and time, wherein an independent variable of the fitting function is time and a dependent variable of the fitting function is the contrast medium concentration.

6. The method according to claim 1, further comprising:
before predicting the time interval from the current time point to the time point when the target concentration value reaches according to the relationship between the contrast medium concentration and time, calculating a difference between a current concentration value of the contrast medium and a concentration value of the contrast medium in a previous scanning; and
determining whether the difference is greater than or equal to a predetermined concentration threshold;
wherein obtaining a relationship between a contrast medium concentration and time according to concentration values of the contrast medium obtained by previous scannings comprises: if the difference is greater than or equal to the predetermined concentration threshold, obtaining the relationship between the contrast medium concentration and time according to concentration values of the contrast medium obtained by previous scannings.

7. The method according to claim 6, further comprising: if the difference is less than the predetermined concentration threshold, continuing performing a next scanning.

8. A device for tracking a contrast medium by scanning, comprising:
a prediction unit, adapted to obtain a relationship between a contrast medium concentration and time according to concentration values of the contrast medium obtained by previous scannings;
a time determination unit, adapted to predict a time interval from a current time point to a time point when a target concentration value reaches according to the relationship between the contrast medium concentration and time; and
a routine examination starting unit, adapted to start a routine examination at the end of the time interval.

9. The device according to claim 8, further comprising:
a first determination unit, adapted to determine whether the time interval from the current time point to the time point when the target concentration value reaches is shorter than a predetermined time threshold after the time interval is predicted according to the relationship between the contrast medium concentration and time;
a scanning termination unit, adapted to stop scanning the contrast medium if the time interval is determined to be shorter than the predetermined time threshold; and
a first scanning continuing unit, adapted to continue performing a next scanning if the time interval is determined to be longer than or equal to the predetermined time threshold.

10. The device according to claim 8, wherein the target concentration value is a concentration value required for the routine examination or any other predetermined concentration value.

11. The device according to claim 9, wherein the predetermined time threshold comprises a scanning period or a plurality of scanning periods.

12. The device according to claim 8, wherein the prediction unit comprises:
an obtaining unit, adapted to obtain concentration values of the contrast medium in previous scannings; and
a fitting unit, adapted to perform fitting to the concentration values of the contrast medium in previous scannings and generate a fitting function between the contrast medium concentration and time, wherein an independent variable of the fitting function is time and a dependent variable of the fitting function is the contrast medium concentration.

13. The device according to claim 8, further comprising:
a difference calculation unit, adapted to calculate a difference between a current concentration value of the contrast medium and a concentration value of the contrast medium in a previous scanning before the time interval from the current time point to the time point when the target concentration value reaches is determined according to the relationship between the contrast medium concentration and time; and
a second determination unit, adapted to determine whether the difference is greater than or equal to a predetermined concentration threshold;
wherein the prediction unit is adapted to obtain a relationship between a contrast medium concentration and time according to concentration values of the contrast medium obtained by previous scannings if the difference is determined to be greater than or equal to the predetermined concentration threshold.

14. The device according to claim 13, further comprising:
a second scanning continuing unit, adapted to continue performing a next scanning when the difference is determined to be less than the predetermined concentration threshold.
